(19) United States
(12) Patent Application Publication
(10) Pub. No.: US 2022/0313115 A1
(43) Pub. Date: Oct. 6, 2022

(54) SYSTEMS AND METHODS FOR A TALKING HEIGHT MEASUREMENT SYSTEM

(71) Applicant: Martin Poulter, Port Lincoln (AU)

(72) Inventor: Martin Poulter, Port Lincoln (AU)

(21) Appl. No.: 17/696,043

(22) Filed: Mar. 16, 2022

Related U.S. Application Data
(60) Provisional application No. 63/167,759, filed on Mar. 30, 2021.

(51) Int. Cl. A61B 5/107; A61B 5/00

(52) U.S. Cl. CPC A61B 5/1072; A61B 5/741; A61B 5/7285; A61B 5/7415; A61B 2562/0257

(57) ABSTRACT

A hands-free height measurement system includes a distance sensor configured to generate a signal indicative of a height of a user, a processor, and a memory. The memory includes instructions stored thereon, which when executed by the processor cause the hands-free height measurement system to receive a first signal, by the distance sensor, indicating a distance from the user to the hands-free height measurement system, compare the indicated distance to a stored predetermined distance from a floor to the hands-free height measurement system, and determine a height of the user based on the comparison.

### CROSS-REFERENCE TO RELATED APPLICATION

The present application claims the benefit of and priority to U.S. Provisional Patent Application Ser. No. 63/167,759, filed on Mar. 30, 2021, the entire content of which is incorporated herein by reference.

### TECHNICAL FIELD

This disclosure relates to a hands-free height measurement system and, more particularly, to structures and methods for a talking hands-free height measurement system.

### SUMMARY

In accordance with aspects of the disclosure, a hands-free height measurement system includes a distance sensor configured to generate a signal indicative of a height of a user, a processor, and a memory. The memory includes instructions stored thereon, which when executed by the processor, cause the hands-free height measurement system to receive a first signal, by the distance sensor, indicating a distance from the user to the hands-free height measurement system, compare the indicated distance to a stored predetermined distance from a floor to the hands-free height measurement system, and determine a height of the user based on the comparison.

In an aspect of the present disclosure, the instructions, when executed by the processor, may further cause the system to provide an audio indication of the height of the user.

In yet another aspect of the present disclosure, the audio indication may include a voice announcing the height of the user.

In an aspect of the present disclosure, the instructions, when executed by the processor, may further cause the system to compare the determined height of the user to a threshold value and provide an audio indication of whether the user is taller than the threshold value based on the comparison.

In another aspect of the present disclosure, the instructions, when executed by the processor, may further cause the system to receive a second signal by the distance sensor, indicating a distance from the floor to the hands-free height measurement system and store the predetermined distance from a floor to the hands-free height measurement system.

In yet another aspect of the present disclosure, the system may further include a motion sensor configured to sense motion of a user in proximity to the hands-free height measurement system.

In a further aspect of the present disclosure, the instructions, when executed by the processor, may further cause the system to detect motion of the user before sensing the first signal and sense the first signal based on the detected motion.

In yet a further aspect of the present disclosure, the system may further include a visual indicator.

In an aspect of the present disclosure, the instructions, when executed by the processor, may further cause the system to provide a visual indication that height measurement has begun.

In another aspect of the present disclosure, the instructions, when executed by the processor, may further cause the system to compare the determined height of the user to a threshold value and provide a visual indication of whether the user is taller than the threshold value or not based on the comparison.

In yet another aspect of the present disclosure, the distance sensor may include an ultrasonic distance sensor, an IR proximity sensor, and/or a laser distance sensor.

In accordance with aspects of the disclosure, a computer-implemented method for hands-free height measurement includes receiving a first signal by a distance sensor of a hands-free height measurement system, the distance sensor configured to generate a signal indicative of a height of a user, the first signal indicating a distance from a user to the hands-free height measurement system; comparing the indicated distance to a stored predetermined distance from a floor to the hands-free height measurement system; and determining a height of the user based on the comparison.

In a further aspect of the present disclosure, the method may further include providing an audio indication of the height of the user.

In accordance with aspects of the disclosure, the audio indication may include a voice and/or a tone.

In an aspect of the present disclosure, the method may further include comparing the determined height of the user to a threshold value and providing an audio indication of whether the user is taller than the threshold value based on the comparison.

In another aspect of the present disclosure, the method may further include receiving a second signal by the distance sensor, indicating a distance from the floor to the hands-free height measurement system, and storing the predetermined distance from a floor to the hands-free height measurement system.

In yet another aspect of the present disclosure, the method may further include detecting motion of the user by a motion sensor configured to sense motion of a user in proximity to the hands-free height measurement system before sensing the first signal and sensing the first signal based on the detected motion.

In a further aspect of the present disclosure, the distance sensor may include an ultrasonic distance sensor, an IR proximity sensor, and/or a laser distance sensor.

In an aspect of the present disclosure, the method may further include providing a visual indication, by a visual indicator, that height measurement has begun.

In another aspect of the present disclosure, the method may further include comparing the determined height of the user to a threshold value and providing a visual indication, by a visual indicator, of whether the user is taller than the threshold value or not based on the comparison.

Other aspects, features, and advantages will be apparent from the description, the drawings, and the claims that follow.

### BRIEF DESCRIPTION OF DRAWINGS

The accompanying drawings, which are incorporated in and constitute a part of this specification, illustrate aspects of the disclosure and, together with a general description of the disclosure given above and the detailed description is given below, explain the principles of this disclosure, wherein:

FIG. 1 is a diagram illustrating a hands-free height measurement system, in accordance with the disclosure, shown while in ues;

FIG. 2 is a block diagram of a controller configured for use with the hands-free height measurement system of FIG. 1, in accordance with the disclosure;

FIG. 3 is a top perspective view of the hands-free height measurement system of FIG. 1, in accordance with the disclosure;

FIG. 4 is a top view of the hands-free height measurement system of FIG. 1, in accordance with the disclosure;

FIG. 5 is a bottom view of the hands-free height measurement system of FIG. 1, in accordance with the disclosure;

FIG. 6 is a top cutaway view of the hands-free height measurement system of FIG. 1, in accordance with the disclosure;

FIG. 7 is a side view of the hands-free height measurement system of FIG. 1, in accordance with the disclosure; and

FIG. 8 is a side cutaway view of the hands-free height measurement system of FIG. 1, in accordance with the disclosure.

### DETAILED DESCRIPTION

Aspects of the disclosed predictive maintenance systems are described in detail with reference to the drawings, in which like reference numerals designate identical or corresponding elements in each of the several views. In addition, directional terms such as front, rear, upper, lower, top, bottom, and the like are used simply for the convenience of description and are not intended to limit the disclosure attached hereto.

In the following description, well-known functions or constructions are not described in detail to avoid obscuring the present disclosure in unnecessary detail.

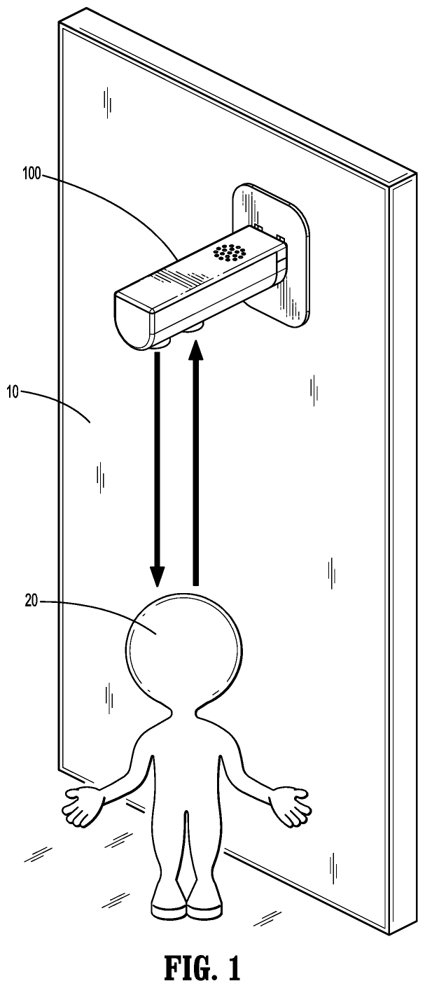

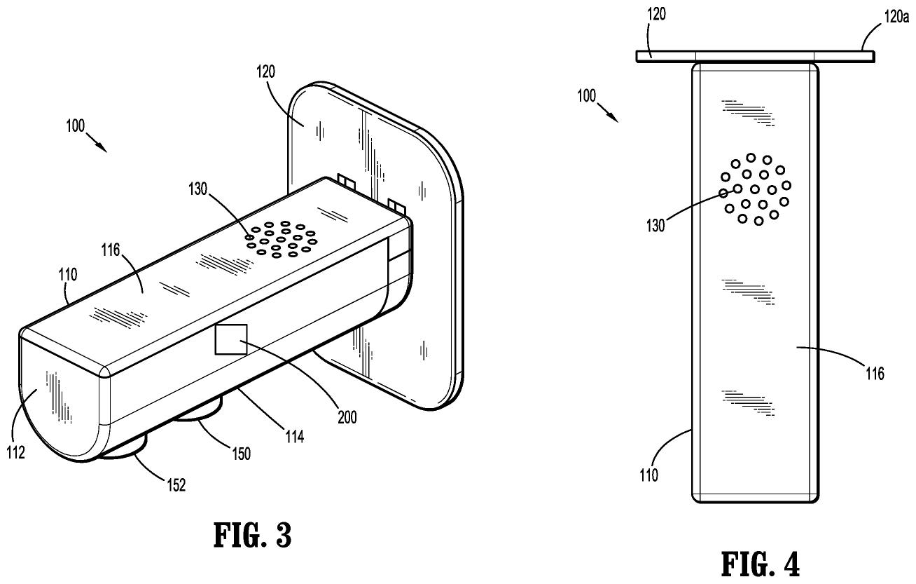

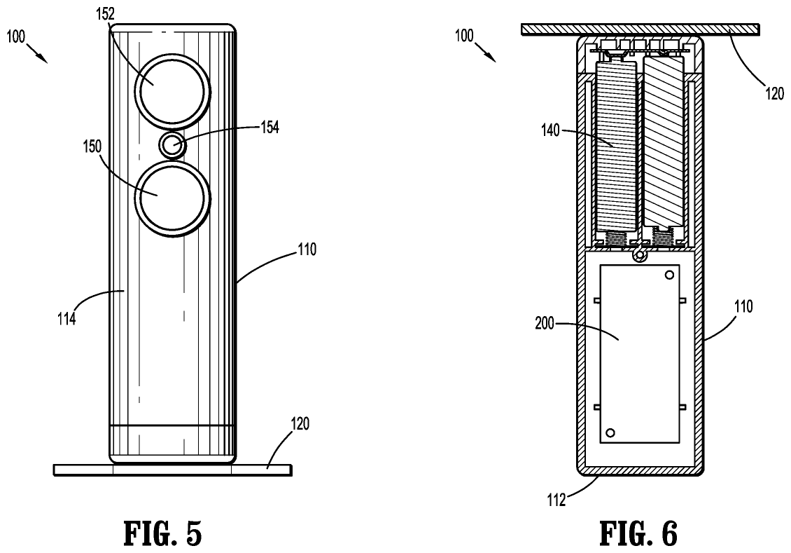

Referring to FIGS. 1 and 3-8, a hands-free height measurement system 100 configured for measurement of a user or subject 20 is shown. The hands-free height measurement system 100 generally includes a body 110, a mounting flange 120, a distance sensor 150 configured to sense a distance from the system 100 (e.g., a bottom-most surface, or ground facing surface) to an object (e.g., such as the user or subject 20 or the floor), a motion sensor 152 configured to sense motion of the user or subject 20, a visual indicator 154 (e.g., a LED), a speaker 132 configured to emit sound (e.g., a voice announcing the measured value), and a controller 200 configured to process sensor signals and determine a height of the user or subject 20.

The body 110 of the hands-free height measurement system 100 generally includes a top surface 116, a bottom surface 114, and an end surface 112. In aspects, the top surface 116 may include a speaker grille 130 configured to protect a speaker 132 and to permit the emission of sound from the speaker 132. In aspects, the distance sensor 150, the motion sensor 152, and the visual indicator 154 may be disposed on the bottom surface 114 of the body 110.

The mounting flange 120 is configured for mounting the hands-free height measurement system 100 to a wall 10 or other surface for mounting (e.g., a mirror, a stand, a door, a post, etc.). The mounting flange 120 may include a suction cup, double-sided adhesive, and/or holes for mounting hardware to enable mounting the hands-free height measurement system 100 at a suitable height (e.g., 2 meters from a floor) for determining the height of the user or subject 20 during operation/use thereof.

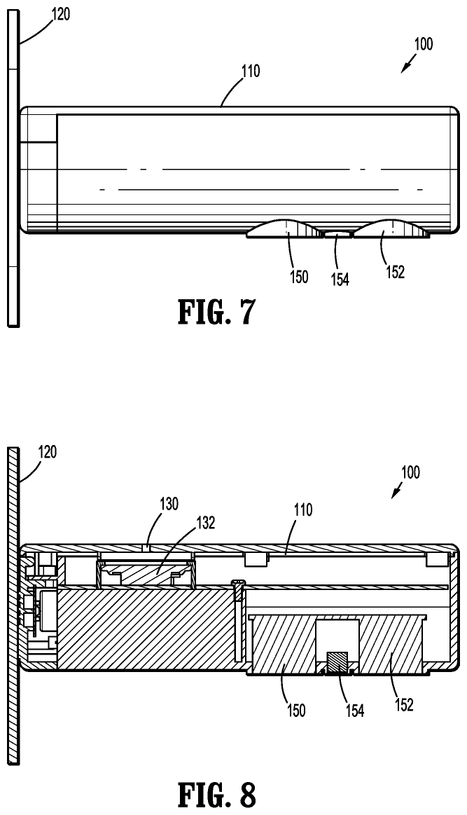

The distance sensor 150 (FIGS. 7 and 8) may include an ultrasonic distance sensor, an IR proximity sensor, and/or a laser distance sensor. The distance sensor 150 functions by outputting a signal (depending on technology; ultrasonic waves, IR, LED, etc.) and measuring the change when the signal returns. Measurement of change can be in the form of the time it takes for a signal to return and/or the intensity of a returned signal.

The motion sensor 152 is configured to detect the presence of a user or subject 20. A detected motion, by the motion sensor 152, is communicated to the controller 200 and may be used to initiate a height measurement of the user or subject 20 when the user or subject 20 enters a sensing zone for motion sensor 152. The motion sensor 152 may include active and/or passive motion sensors.

The visual indicator 154 is configured to provide a visual indication of detected motion and/or provide an indication that the system 100 is determining a height of the user or subject 20. The visual indicator 154 may also provide a visual indication that the height of the user or subject 20 is above a threshold value. For example, the visual indicator 154 may turn red if the height of the user or subject 20 is below the threshold value and green if the height of the user or subject 20 is above the threshold value. Further, the visual indicator 154 may display alpha-numeric charaters (e.g., distance in metric or non-metric/English units) to convey to the public the height information measured.

The speaker 132 is configured to emit a sound. For example, the controller 200 may communicate a signal to the speaker 132 to indicate the height of the user or subject 20 and/or whether a user or subject 20 is taller than a predetermined height (e.g., a voice announcing the measured value). The indication may include a tone (e.g., a beep) and/or a voice.

In aspects, the system 100 may be mounted at a predetermined height, for example, at two meters high, and the predetermined height may be stored by the controller 200 as a calibration distance. In aspects, the system 100 may be calibrated by measuring a distance from a bottom-most surface of the system 100 to the ground and storing the measured distance as the calibration distance.

In use, the controller 200 is configured to subtract a measured height/distance from the stored calibration distance. For example, a measure/distance from the top of a head of a user or subject 20 and subtract the measured distance to the the head of the user or subject 20 from the distance to the ground. For example, if the system 100 is mounted seven feet (e.g., 7.0 feet) high from the ground, when calibrated the controller 200 measures and stores the measured distance from the ground (e.g., 7.0 feet), then a user or subject 20 would stand under the system 100 and the motion sensor 152 would detect the motion of the user or subject 20 and cause the controller 200 to measure the distance to the top of the head of the user or subject 20 (e.g., about 2.0 feet). The controller 200 would then subtract the two-foot (2.0 feet) distance (e.g., distance to the top of the head of the user or subject 20) from the seven-foot (7.0 feet) distance (e.g., distance to the ground) and calculate that the user or subject 20 is five feet (5.0 feet) tall. Then the controller 200 provides audio notification, for example, by announcing the height of the user or subject 20, e.g., “you are five feet tall,” or “five feet, zero inches.”

In aspects, the system 100 may be used in amusement parks for determining if a user or subject 20 meets the height requirements for a ride or attraction. The controller 200 may compare the measured height to a predetermined number or threshold values (e.g., the height requirement for a ride) and provide an audio indication as to whether the user or subject 20 meets and/or exceeds the predetermined number. For example, the audio notification may announce “pass” or “fail,” or “you meet/do not meet the requirements to ride this ride.”

In aspects, the hands-free height measurement system 100 may be powered by batteries 140 or may be AC powered.

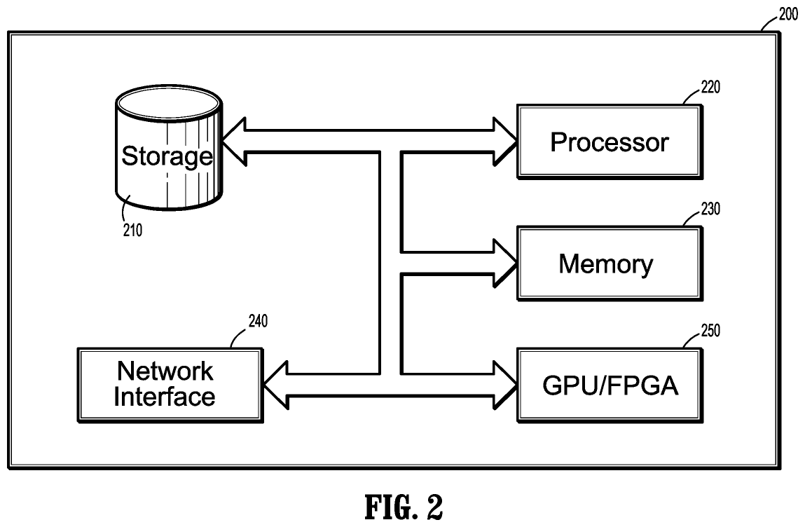

FIG. 2 illustrates that controller 200 includes a processor 220 connected to a computer-readable storage medium or a memory 230. The computer-readable storage medium or memory 230 may be a volatile type of memory, e.g., RAM, or a non-volatile type of memory, e.g., flash media, disk media, etc. In various aspects of the disclosure, the processor 220 may be another type of processors such as a digital signal processor, a microprocessor, an ASIC, a graphics processing unit (GPU), a field-programmable gate array (FPGA), or a central processing unit (CPU). In certain aspects of the disclosure, network inference may also be accomplished in systems that have weights implemented as memristors, chemically, or other inference calculations, as opposed to processors.

In aspects of the disclosure, the memory 230 can be random access memory, read-only memory, magnetic disk memory, solid-state memory, optical disc memory, and/or another type of memory. In some aspects of the disclosure, the memory 230 can be separate from the controller 200 and can communicate with the processor 220 through communication buses of a circuit board and/or through communication cables such as serial ATA cables or other types of cables. The memory 230 includes computer-readable instructions that are executable by the processor 220 to operate the controller 200. In other aspects of the disclosure, the controller 200 may include a network interface 240 to communicate with other computers or to a server. A storage device 210 may be used for storing data.

The disclosed method may run on the controller 200 or on a user device, including, for example, on a mobile device, an IoT device, or a server system.

Moreover, the disclosed structure can include any suitable mechanical, electrical, and/or chemical components for operating the disclosed pivot predictive maintenance system or components thereof. For instance, such electrical components can include, for example, any suitable electrical and/or electromechanical and/or electrochemical circuitry, which may include or be coupled to one or more printed circuit boards. As used herein, the term “controller” includes “processor,” “digital processing device,” and like terms, and are used to indicate a microprocessor or central processing unit (CPU). The CPU is the electronic circuitry within a computer that carries out the instructions of a computer program by performing the basic arithmetic, logical, control and input/output (I/O) operations specified by the instructions, and by way of non-limiting examples, include server computers. In some aspects, the controller includes an operating system configured to perform executable instructions. The operating system is, for example, software, including programs and data, which manages hardware of the disclosed surgical stapling apparatus and provides services for execution of applications for use with the disclosed surgical stapling apparatus. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. In some aspects, the operating system is provided by cloud computing.

In some aspects, the term “controller” may be used to indicate a device that controls the transfer of data from a computer or computing device to a peripheral or separate device and vice versa, and/or a mechanical and/or electromechanical device (e.g., a lever, knob, etc.) that mechanically operates and/or actuates a peripheral or separate device.

In aspects, the controller includes a storage and/or memory device. The storage and/or memory device is one or more physical apparatus used to store data or programs on a temporary or permanent basis. In some aspects, the controller includes volatile memory and requires power to maintain stored information. In various aspects, the controller includes non-volatile memory and retains stored information when it is not powered. In some aspects, the non-volatile memory includes flash memory. In certain aspects, the non-volatile memory includes dynamic random-access memory (DRAM). In some aspects, the non-volatile memory includes ferroelectric random-access memory (FRAM). In various aspects, the non-volatile memory includes phase-change random access memory (PRAM). In certain aspects, the controller is a storage device including, by way of non-limiting examples, CD-ROMs, DVDs, flash memory devices, magnetic disk drives, magnetic tapes drives, optical disk drives, and cloud computing-based storage. In various aspects, the storage and/or memory device is a combination of devices such as those disclosed herein.

In some aspects, the controller includes a display to send visual information to a user. In various aspects, the display is a cathode ray tube (CRT). In various aspects, the display is a liquid crystal display (LCD). In certain aspects, the display is a thin film transistor liquid crystal display (TFT-LCD). In aspects, the display is an organic light emitting diode (OLED) display. In certain aspects, on OLED display is a passive-matrix OLED (PMOLED) or active-matrix OLED (AMOLED) display. In aspects, the display is a plasma display. In certain aspects, the display is a video projector. In various aspects, the display is interactive (e.g., having a touch screen or a sensor such as a camera, a 3D sensor, a LiDAR, a radar, etc.) that can detect user interactions/gestures/responses and the like. In some aspects, the display is a combination of devices such as those disclosed herein.

The controller may include or be coupled to a server and/or a network. As used herein, the term “server” includes “computer server,” “central server,” “main server,” and like terms to indicate a computer or device on a network that manages the surgical stapling apparatus, components thereof, and/or resources thereof. As used herein, the term “network” can include any network technology including, for instance, a cellular data network, a wired network, a fiber optic network, a satellite network, and/or an IEEE 802.11a/b/g/n/ac wireless network, among others.

In various aspects, the controller can be coupled to a mesh network. As used herein, a “mesh network” is a network topology in which each node relays data for the network. All mesh nodes cooperate in the distribution of data in the network. It can be applied to both wired and wireless networks. Wireless mesh networks can be considered a type of “Wireless ad hoc” network. Thus, wireless mesh networks are closely related to Mobile ad hoc networks (MANETs). Although MANETs are not restricted to a specific mesh network topology, Wireless ad hoc networks or MANETs can take any form of network topology. Mesh networks can relay messages using either a flooding technique or a routing technique. With routing, the message is propagated along a path by hopping from node to node until it reaches its destination. To ensure that all its paths are available, the network must allow for continuous connections and must reconfigure itself around broken paths, using self-healing algorithms such as Shortest Path Bridging. Self-healing allows a routing-based network to operate when a node breaks down or when a connection becomes unreliable. As a result, the network is typically quite reliable, as there is often more than one path between a source and a destination in the network. This concept can also apply to wired networks and to software interaction. A mesh network whose nodes are all connected to each other is a fully connected network.

In some aspects, the controller may include one or more modules. As used herein, the term “module” and like terms are used to indicate a self-contained hardware component of the central server, which in turn includes software modules. In software, a module is a part of a program. Programs are composed of one or more independently developed modules that are not combined until the program is linked. A single module can contain one or several routines, or sections of programs that perform a particular task.

As used herein, the controller includes software modules for managing various aspects and functions of the disclosed surgical stapling apparatus or components thereof.

The disclosed structure may also utilize one or more controllers to receive various information and transform the received information to generate an output. The controller may include any type of computing device, computational circuit, or any type of processor or processing circuit capable of executing a series of instructions that are stored in memory. The controller may include multiple processors and/or multicore central processing units (CPUs) and may include any type of processor, such as a microprocessor, digital signal processor, microcontroller, programmable logic device (PLD), field programmable gate array (FPGA), or the like. The controller may also include a memory to store data and/or instructions that, when executed by the one or more processors, cause the one or more processors to perform one or more methods and/or algorithms.

Any of the herein described methods, programs, algorithms or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

As can be appreciated, securement of any of the components of the disclosed apparatus can be effectuated using known securement techniques such welding, crimping, gluing, fastening, etc.

Persons skilled in the art will understand that the structures and methods specifically described herein and illustrated in the accompanying figures are non-limiting exemplary aspects, and that the description, disclosure, and figures should be construed merely as exemplary of particular aspects. It is to be understood, therefore, that this disclosure is not limited to the precise aspects described, and that various other changes and modifications may be effectuated by one skilled in the art without departing from the scope or spirit of the disclosure. Additionally, it is envisioned that the elements and features illustrated or described in connection with one exemplary aspect may be combined with the elements and features of another without departing from the scope of this disclosure, and that such modifications and variations are also intended to be included within the scope of this disclosure. Indeed, any combination of any of the disclosed elements and features is within the scope of this disclosure. Accordingly, the subject matter of this disclosure is not to be limited by what has been particularly shown and described.

What is claimed is:

1. A hands-free height measurement system, comprising:
a distance sensor configured to generate a signal indicative of a height of a user;
a processor; and
a memory, including instructions stored thereon, which when executed by the processor cause the hands-free height measurement system to:
receive a first signal, by the distance sensor, indicating a distance from the user to the hands-free height measurement system;
compare the indicated distance to a stored predetermined distance from a floor to the hands-free height measurement system; and
determine a height of the user based on the comparison.

2. The hands-free height measurement system of claim 1, wherein the instructions, when executed by the processor, further cause the system to provide an audio indication of the height of the user.

3. The hands-free height measurement system of claim 2, wherein the audio indication includes a voice announcing the height of the user.

4. The hands-free height measurement system of claim 1, wherein the instructions, when executed by the processor, further cause the system to:
compare the determined height of the user to a threshold value; and
provide an audio indication of whether the user is taller than the threshold value based on the comparison.

5. The hands-free height measurement system of claim 1, wherein the instructions, when executed by the processor, further cause the system to:
receive a second signal, by the distance sensor, indicating a distance from the floor to the hands-free height measurement system; and
store the predetermined distance from a floor to the hands-free height measurement system.

6. The hands-free height measurement system of claim 1, further comprising a motion sensor configured to sense motion of a user in proximity to the hands-free height measurement system.

7. The hands-free height measurement system of claim 6, wherein the instructions, when executed by the processor, further cause the system to:
detect motion of the user before sensing the first signal; and
sense the first signal based on the detected motion.

8. The hands-free height measurement system of claim 1, further comprising a visual indicator.

9. The hands-free height measurement system of claim 8, wherein the instructions, when executed by the processor, further cause the system to provide a visual indication that height measurement has begun.

10. The hands-free height measurement system of claim 8, wherein the instructions, when executed by the processor, further cause the system to:
compare the determined height of the user to a threshold value; and
provide a visual indication of whether the user is taller than the threshold value or not based on the comparison.

11. The hands-free height measurement system of claim 1, wherein the distance sensor includes at least one of an ultrasonic distance sensor, an IR proximity sensor, or a laser distance sensor.

12. A computer-implemented method for hands-free height measurement, the computer-implemented method comprising:
receiving a first signal by a distance sensor of a hands-free height measurement system, the distance sensor configured to generate a signal indicative of a height of a user, the first signal indicating a distance from a user to the hands-free height measurement system;
comparing the indicated distance to a stored predetermined distance from a floor to the hands-free height measurement system; and
determining a height of the user based on the comparison.

13. The computer-implemented method of claim 12, further comprising providing an audio indication of the height of the user.

14. The computer-implemented method of claim 13, wherein the audio indication includes a voice and/or a tone.

15. The computer-implemented method of claim 12, further comprising:
comparing the determined height of the user to a threshold value; and
providing an audio indication of whether the user is taller than the threshold value based on the comparison.

16. The computer-implemented method of claim 12, further comprising:
receiving a second signal, by the distance sensor, indicating a distance from the floor to the hands-free height measurement system; and
storing the predetermined distance from a floor to the hands-free height measurement system.

17. The computer-implemented method of claim 16, further comprising:
detecting motion of the user by a motion sensor configured to sense motion of a user in proximity to the hands-free height measurement system before sensing the first signal; and
sensing the first signal based on the detected motion.

18. The computer-implemented method of claim 12, wherein the distance sensor includes at least one of an ultrasonic distance sensor, an IR proximity sensor, or a laser distance sensor.

19. The computer-implemented method of claim 12, further comprising providing a visual indication, by a visual indicator, that height measurement has begun.

20. The computer-implemented method of claim 12, further comprising:
comparing the determined height of the user to a threshold value; and
providing a visual indication, by a visual indicator, of whether the user is taller than the threshold value or not based on the comparison.
